(19) United States
(12) Patent Application Publication
(10) Pub. No.: US 2023/0301637 A1
(43) Pub. Date: Sep. 28, 2023

(54) APPARATUS, DEVICE AND METHOD FOR COLLECTING SAMPLE FLUID

(71) Applicant: Summa Finland Oy, Oulu (FI)

(72) Inventors: Wojciech Kurek, Wroclaw (PL); Marko Mäkinen, Oulu (FI); Juuso Nissilä, Oulu (FI)

(73) Assignee: Summa Finland Oy, Oulu (FI)

(21) Appl. No.: 18/124,631

(22) Filed: Mar. 22, 2023

(30) Foreign Application Priority Data
Mar. 24, 2022 (FI) FI20225260

(51) Int. Cl. A61B 10/00

(52) U.S. Cl. CPC A61B 10/0045; A61B 2010/009

(57) ABSTRACT

Disclosed is an apparatus for collecting a sample fluid including a hollow elongate body having a volume. The volume has a bottom and an inlet opposite to the bottom of the volume, wherein the volume of the hollow elongate body is configured to receive the sample fluid via the inlet. The apparatus further comprising at least one sensor, coupled to the hollow elongate body, wherein the at least one sensor has at least one opening for receiving at least a part of the sample fluid from the volume. The apparatus further comprising a plurality of electrical connectors, arranged with the hollow elongate body, connected to the at least one sensor.

### TECHNICAL FIELD

The present disclosure relates, generally, to analytical devices. More specifically, the present disclosure relates to an apparatus, a device and a method for collecting of a sample fluid.

### BACKGROUND

Generally, fluids from subjects are collected for various applications such as molecular epidemiology, clinical trial and basic research studies. Examples of such fluids include blood, urine, saliva, or snot. Many medical advances, including studies of heart disease, AIDS and cancer, have resulted from preliminary developmental studies are dependent on appropriate collection and precise use of these fluids.

Conventionally, such fluids may be collected by using lateral flow devices. Typically, the lateral flow device is a simple device intended to detect the presence of a target substance in a liquid sample without the need for any specialized and costly equipment. However, a limitation of the existing solutions is that the collection and analysis of the fluid is not accurate and reliable. Moreover, despite, the affordable and less resource extensive nature of the existing solutions, these solutions fail to analyze several properties from the collected sample of the fluid.

Therefore, in light of the foregoing discussion, there exists a need to overcome the aforementioned drawbacks associated with known techniques for collecting a sample fluid.

### SUMMARY

The present disclosure seeks to provide an apparatus for collecting a sample fluid. The present disclosure also seeks to provide a device for collecting a sample fluid. The present disclosure also seeks to provide a method for collecting a sample fluid. An aim of the present disclosure is to provide a solution that overcomes at least partially the problems encountered in the prior art.

In one aspect, an embodiment of the present disclosure seeks to provide an apparatus for collecting a sample fluid, the apparatus comprising:
a hollow elongate body having a volume, comprising a bottom and an inlet opposite to the bottom of the volume, wherein the volume of the hollow elongate body is configured to receive the sample fluid via the inlet;
at least one sensor, coupled to the hollow elongate body, wherein the at least one sensor has at least one opening for receiving at least a part of the sample fluid from the volume; and
a plurality of electrical connectors, arranged with the hollow elongate body, connected to the at least one sensor.

In another aspect, an embodiment of the present disclosure seeks to provide a device for collecting a sample fluid, the device comprising:
an apparatus for collecting the sample fluid, wherein the apparatus is the aforementioned apparatus; and
a reader part having a plurality of base connectors to connect with a plurality of electrical connectors of at least one sensor of the apparatus, and a controller configured to
   provide an electrical signal to the at least one sensor and read a related electrical signal from the at least one sensor via the plurality of base connectors, and
   use the provided and related electrical signal to form an indicator value of an amount of at least one molecule of interest in the sample fluid.

In yet another aspect, an embodiment of the present disclosure seeks to provide a method for collecting a sample fluid, the method comprising:
collecting the sample fluid in a volume of a hollow elongate body of an apparatus via an inlet of the hollow elongate body;
delivering at least a part of the sample fluid from the volume of the hollow elongate body to the at least one sensor coupled to the hollow elongate body, wherein at least a part of the sample fluid is delivered to the at least one sensor by creating a pressure difference between the volume and the at least one sensor; and
generating an indicator value indicating an amount of at least one molecule of interest in the sample fluid.

Embodiments of the present disclosure substantially eliminate or at least partially address the aforementioned problems in the prior art, and provide an efficient, reliable, and accurate way of collecting and analyzing sample fluids. Beneficially, the apparatus ensures an adequate amount of the sample fluid to be collected for an accurate and precise analysis thereof. Moreover, the disclosed apparatus and the device maybe used in medical applications, doping sports, health support areas, and so forth.

Additional aspects, advantages, features and objects of the present disclosure will be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those skilled in the art will understand that the drawings are not to scale. Wherever possible, like elements have been indicated by identical numbers.

Embodiments of the present disclosure will now be described, by way of example only, with reference to the following diagrams wherein:

FIG. 1A is a perspective view of an apparatus for collecting a sample fluid, in accordance with an embodiment of the present disclosure;

FIG. 1B is an enlarged view of at least one sensor coupled with the hollow elongate body, in accordance with an embodiment of the present disclosure;

FIGS. 2A and 2B are schematic illustrations of at least one opening of at least one sensor coupled with the hollow elongate body, in accordance with different embodiments of the present disclosure;

FIG. 2C is a cross-sectional view of FIG. 2B, at least one sensor is coupled to a hollow elongate body, in accordance with an embodiment of the present disclosure;

FIG. 3 is a perspective view of a coupling between the hollow elongate body and the least one sensor, in accordance with another embodiment of the present disclosure;

FIG. 4 is a layout of polygonal sides printed side by side, in accordance with an embodiment of the present disclosure;

FIG. 5 is a perspective view of a device for collecting a sample fluid, in accordance with an embodiment of the present disclosure;

FIG. 6 is a perspective view of a device for collecting a sample fluid, in accordance with another embodiment of the present disclosure;

FIG. 7 is a perspective view depicting the engagement of a hollow elongate body of an apparatus with a reader part, in accordance with an embodiment of the present disclosure;

FIG. 8 is an implementation environment of a device for collecting a sample fluid, in accordance with an embodiment of the present disclosure; and

FIG. 9 illustrates a flowchart depicting steps of a method for collecting a sample fluid, in accordance with an embodiment of the present disclosure.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practicing the present disclosure are also possible.

In one aspect, an embodiment of the present disclosure provides an apparatus for collecting a sample fluid, the apparatus comprising:
a hollow elongate body having a volume, comprising a bottom and an inlet opposite to the bottom of the volume, wherein the volume of the hollow elongate body is configured to receive the sample fluid via the inlet;
at least one sensor, coupled to the hollow elongate body, wherein the at least one sensor has at least one opening for receiving at least a part of the sample fluid from the volume; and
a plurality of electrical connectors, arranged with the hollow elongate body, connected to the at least one sensor.

In another aspect, an embodiment of the present disclosure seeks to provide a device for collecting a sample fluid, the device comprising:
an apparatus for collecting the sample fluid, wherein the apparatus is the aforementioned apparatus; and
a reader part having a plurality of base connectors to connect with a plurality of electrical connectors of at least one sensor of the apparatus, and a controller configured to
   provide an electrical signal to the at least one sensor and read a related electrical signal from the at least one sensor via the plurality of base connectors, and
   use the provided and related electrical signal to form an indicator value of an amount of at least one molecule of interest in the sample fluid.

In yet another aspect, an embodiment of the present disclosure seeks to provide a method for collecting a sample fluid, the method comprising:
collecting the sample fluid in a volume of a hollow elongate body of an apparatus via an inlet of the hollow elongate body;
delivering at least a part of the sample fluid from the volume of the hollow elongate body to the at least one sensor coupled to the hollow elongate body, wherein at least a part of the sample fluid is delivered to the at least one sensor by creating a pressure difference between the volume and the at least one sensor; and
generating an indicator value indicating an amount of at least one molecule of interest in the sample fluid.

The generated indicator value can be used to analyze the sample fluid. As an example the indicator value can represent amount of certain molecule such as hormone in the sample.

Herein, the apparatus of the present disclosure aims to be more suitable and efficient for collecting the sample fluid, i.e., a biological material such as saliva, blood, snot or urine collected from a subject's body. The apparatus of the present disclosure is designed to ensure that an adequate amount of the sample fluid is collected via the apparatus to facilitate an accurate and precise analysis of the sample fluid, when required, using the aforementioned device using the aforementioned method.

The apparatus comprises a hollow elongate body having a volume. Herein, the term “hollow elongate body” refers to a body having a circumferential wall defined over a certain length between two or more ends (namely, the bottom and an inlet opposite to the bottom) thereof, enclosing a hollow space therebetween. Herein, the hollow space forms the “volume” of the hollow elongate body for receiving the sample fluid therein via the inlet. The inlet serves as a hollow opening at an end opposite to the bottom that covers the hollow elongate body entirely at one end.

Optionally, a cross-section of the hollow elongate body may be of a round shape, a polygonal shape (such as hexagonal, triangular prism shapes, cuboidal shape, and so on) or any other suitable shape. Moreover, based on the cross-section of the hollow elongate body, the hollow elongate body may comprise one or more sides arranged between the bottom and the inlet of the hollow elongate body. In an example, for a hexagonal cross-section, the hollow elongate body has 6 sides.

Moreover, the apparatus comprises at least one sensor, coupled to the hollow elongate body, wherein the at least one sensor has at least one opening for receiving the sample fluid from the volume. The term “sensor” refers to a device configured to sense data related to a certain physical, electrical, biological, or chemical property. Optionally, the sensor may be positioned close to the bottom of the hollow elongate body. Optionally, alternatively, the sensor may be positioned partway along the length of the hollow elongate body between the bottom and the inlet. Optionally, the at least one sensor may be coupled on an inner surface (i.e. the surface forming the volume) of at least one side of the hollow elongate body. Optionally, the at least one sensor may be coupled on an outer surface (i.e. the surface open to an external environment) of at least one side of the hollow elongate body.

The at least one opening of the at least one sensor enables the at least one sensor to use the received at least a part of the sample fluid to efficiently sense the data corresponding thereto. Optionally, the at least one opening of the at least one sensor may be implemented as holes, microfluidic channels, or a porous membrane. Herein, the “microfluidic channels” may refer to a plurality of thin channels that are designed to restrict the flow of the at least part of the sample fluid to flow into the at least one sensor from the volume of the hollow elongate body. In deed microflow channels can be arranged to allow (by defining a cross section of channels) adequate amount of fluid to enter into the at least one sensor in a controlled manner. The “porous membrane” may refer to a membrane with small holes defined over the surface of the membrane that allow the at least one sensor to receive at least a part of the sample fluid from the volume of the hollow elongate body.

Optionally, the at least one sensor is an electrochemical sensor adapted to change its electrical characters as a function of an amount of a first molecule of interest in the sample fluid. Herein, the “first molecule of interest” relates to a molecule present in the sample fluid whose one or more properties are to be sensed via the at least one sensor. Optionally, the first molecule of interest may be a hormone, a protein, a salt, a chemical, and the like. Herein, the “electrochemical sensor” refers to a sensor that changes its electrical character on coming in contact with a certain particle or molecule, namely the first molecule of interest. In essence, the electrochemical sensor, upon contacting the first molecule of interest present in the sample fluid, brings in a change in its electrical characters. It will be appreciated that the change in electrical characters is dependent on the amount of the first molecule of interest in the sample fluid. In an example, if the amount of the first molecule of interest is high, the change in the electrical characters of the electrochemical sensors is also high. In another example, if the amount of the first molecule of interest is low, the change in the electrical characters of the electrochemical sensors is also low.

In an embodiment, number of electrochemical sensors is at least two, of which a first one is adapted to change its electrical characters as the function of the amount of the first molecule of interest in the sample fluid and a second one is adapted to change its electrical characters as a function of an amount of a second molecule of interest in the sample fluid, and wherein the first molecule is different from the second molecule. In essence, the sample fluid may contain more than one molecule of interest, such as the first molecule of interest, the second molecule of interest, and so on, wherein the first molecule of interest brings in the change of electrical characters in the first electrochemical sensor, the second molecule of interest brings in the change of electrical characters in the second electrochemical sensor, and so on. In an example, the first molecule of interest may be a hormone, such as thyroxine, testosterone, cortisol, and so on, and the second molecule of interest may be a salt, such as potassium, magnesium, and so on. In an other example the first molecule of interest may be a first hormone such as testosterone and the second molecule of interest may be a second hormone such as cortisol. This set up of measuring two (or more) molecules of interest brings technical benefit of enabling to measure relative amount of the molecules of interest as function of time for example.

Furthermore, the apparatus comprises a plurality of electrical connectors, arranged with the hollow elongate body, connected to the at least one sensor. Herein, the “plurality of electrical connectors” refers to the electrical wiring connections configured to transmit electrical signals, from an external electrical source, to the at least one sensor. The plurality of electrical connectors is installed in the apparatus to power the at least one sensor, thus, enabling the at least one sensor to carry out its function of data collection and optionally analysis thereof. In this regard, the plurality of electrical connectors are coupled to the at least one sensor. Optionally, the plurality of electrical connectors are arranged on the outer surface of at least one side of the hollow elongate body.

Optionally, the apparatus further comprises an insertable part configured to be inserted at least partly into the volume of the hollow elongate body, through the inlet thereof, to deliver the sample fluid to the at least one sensor. Herein, the “insertable part” refers to a part that may be inserted either partially or completely into the volume of the hollow elongate body through the inlet thereof, to aid in delivering the sample into the volume of the hollow elongate body and subsequently to the at least one sensor. Beneficially, the insertable part ensures enough fluid is led to the at least one sensors.

Optionally, the insertable part comprises a porous tissue for collecting the sample fluid. Notably, the porous tissue comprises plurality of holes therein for collecting the sample fluid therein. In essence, the insertable part having the sample fluid collected therein, is then inserted either partially or completely into the volume of the hollow elongate body, in order to align the insertable part with the bottom of the hollow elongate body such that the insertable part can deliver at least a part of the sample fluid to the at least one sensor via the at least one opening therein. Optionally, the sample fluid may be collected in the insertable part after the insertable part is inserted either partly or completely into the volume of the hollow elongate body. In such case, there may be a locking clip or a similar mechanism between the insertable part and the hollow elongate body. Alternatively, optionally, the sample fluid may be collected in the insertable part before the insertable part is inserted either partly or completely into the volume of the hollow elongate body. When the apparatus is used the insertable part is typically first placed in a cavity of a user to collect sample fluid from the user. As an example the insertable part can be said porous tissue which is placed in mouth of the user. The tissue can be placed for example between lip and tooth of the user for period of time of few seconds up to few minutes. During said time the insertable part absorbs from mouth saliva. The saliva (with possible hormones to be measured) can be then delivered to the apparatus by inserting the insertable part in the hollow elongate body via the inlet. Advantage of this is that collecting sample from a user is easy.

Alternatively, the insertable part comprises a hard surface configured to push onwards the sample fluid into the volume of the hollow elongate body and subsequently to the at least one sensor.

Alternatively, the sample fluid may be provided to the at least one sensor directly, such as by dropping the sample fluid in the volume of the hollow elongate body closer to the bottom thereof or closer to the at least one sensor. Optionally, in this case, the sample fluid may be dropped using ways known to a person skilled in the art, such as for example by using a dropper, a syringe, a swab, and so forth.

Optionally, the apparatus further comprises a pressing part configured to drive the insertable part into the volume of the hollow elongate body, and to change pressure inside of the volume to deform the insertable part to deliver the sample fluid to the at least one sensor. Herein, the “pressing part” refers to a part that when in use presses or exerts a pressure or force over the insertable part, thus, driving the insertable part into the volume of the hollow elongate body. Beneficially, the pressing part is configured to drive the insertable part tightly against the bottom of the hollow elongate body to ensure that enough sample fluid is supplied from the insertable part to the at least one sensor. Moreover, the driving of the insertable part into the volume creates a change in pressure inside the volume that results in the deforming of the insertable part. The deforming of the insertable part allows at least a part of the collected sample fluid inside the insertable part to flow out and pass into the at least one sensor through the at least one opening. In essence, the inserting part is driven into the volume of the hollow elongate body via the pressing part, such that to deform the inserting part to deliver at least a part of the sample fluid to the at least one sensor. Example of the pressing part is a piston.

Optionally, the pressing part is attached to the insertable part. Optionally, the pressing part may be permanently attached to the insertable part to form one single part, such that the pressing part serves as a grip for the insertable part to be driven either partially or completely into the volume of the hollow elongate body. Optionally, the pressing part may be fabricated from a same fabrication material like a fabrication material of the insertable part, so that the insertable part and the pressing part may be made as a single piece.

Moreover, the present disclosure also relates to the device as described above. Various embodiments and variants disclosed above apply mutatis mutandis to the device.

The device comprises a reader part having a plurality of base connectors to connect with a plurality of electrical connectors of at least one sensor of the apparatus. The term “reader part” as used herein refers to a receiver part for receiving the hollow elongate body therein, and configured to, when in use, read the information received from the at least one sensor. Herein, the term “plurality of base connectors” relates to electrical wiring arrangement installed in the reader part of the device with the purpose of providing electricity to the plurality of electrical connectors of at least one sensor of the apparatus.

Moreover, the base connectors connect with the controller configured to provide an electrical signal to the at least one sensor and read a related electrical signal from the at least one sensor via the plurality of base connectors. In essence, the electrical signal that is being supplied to the at least one sensor via the plurality of base connectors is provided by the controller. Furthermore, the data measured by the at least one sensor is sent to the controller via the plurality of base connectors in the form of related electrical signals. Herein, the “related electrical signal” may preferably relate to signals corresponding to the change in electrical characters measured as the function of the amount of the first molecule of interest by the at least one sensor. Notably, the related electrical signal are in a form readable by the controller.

Furthermore, the controller is configured to use the provided and related electrical signal to form an indicator value of an amount of at least one molecule of interest in the sample fluid. In essence, upon receiving the related electrical signal, the controller initiates a comparison between the electrical signal that is provided by the controller and the related electrical signal which is received the controller, where the comparison would relate to the amount of the first molecule of interest in the sample fluid. Based on the comparison, the indicator value indicating the amount of the first molecule of interest present in the sample fluid is generated.

Optionally, the device comprises a communication interface to provide the indicator value to a computing unit, wherein the computing unit is configured to analyse the indicator value to determine a significance of the amount of the at least one molecule in the sample fluid. Herein, the “computing unit” may relate to an external cloud server, a database or a processor. The reader part may transfer the information received from the at least one sensor to the computing device for further processing and analysis of the information. Optionally, the computing unit may include, but is not limited to, cellular phones, personal digital assistants (PDAs), handheld devices, wireless modems, laptop computers, personal computers, and so forth. Notably, the computing unit receives the indicator value from the device over the communication interface established between the device and the computing unit. Subsequently, the received indicator value is analysed by the computing unit, enabling determining the significance of the amount of the at least one molecule of interest in the sample fluid. Herein, the at least one molecule of interest may include the first molecule of interest, the second molecule of interest as measured by the first sensor and the second sensor, respectively. Optionally, the communication interface is a wireless or wired communication unit. Optionally, the communication interface may include, but is not limited to, Bluetooth®, Wireless Fidelity (Wi-Fi), Local Area Networks (LANs), Wide Area Networks (WANs), Metropolitan Area Networks (MANs), Wireless LANs (WLANs), Wireless WANs (WWANs), Wireless MANs (WMANs), the Internet, second generation (2G) telecommunication networks, third generation (3G) telecommunication networks, fourth generation (4G) telecommunication networks, fifth generation (5G) telecommunication networks and Worldwide Interoperability for Microwave Access (WiMAx) networks.

Optionally, the device further comprises a measurement arrangement configured to measure an amount of sample fluid supplied to the at least one sensor and use the measured amount of the sample fluid to provide an instruction to a user of the device. Herein, the “measurement arrangement” may relate to sensor, such as a humidity sensor, that is installed with the purpose of measuring the humidity inside the at least one sensor, where the value of the measured humidity determines the amount of the sample fluid supplied to the at least once sensor for an efficient analysis thereof. Once the humidity inside the at least one sensor exceeds a certain predefined amount, the measurement arrangement may indicate that an adequate amount of the sample fluid has been provided to the at least one sensor. Thus, based on the indication from the measurement arrangement, the device provides the instruction if more sample fluid needs to be collected or not, to the user of the device. For example, if the measurement arrangement indicates that the sample fluid supplied is less that the adequate amount, the device may use an LED configured to blink red light, informing the user that more sample fluid needs to be collected. Similarly, if the measurement arrangement indicates that the sample fluid supplied is more that the adequate amount, the device may use an LED configured to blink green light, informing the user that adequate amount of sample fluid is collected. Additional benefit of said measurement sensor is to know if more force (pressure) should be applied to the insertable part with the pressing part.

Moreover, the present disclosure also relates to the method as described above. Various embodiments and variants disclosed above apply mutatis mutandis to the method.

Optionally, the collecting further comprises:
contacting an insertable part with the sample fluid;
inserting the insertable part into the volume of the hollow elongate body via an inlet of the hollow elongate body to create a pressure difference between the volume and the at least one sensor; and
deforming the insertable part with the created pressure difference to deliver at least a part of the sample fluid to the at least one sensor. As an example of contacting of the insertable part with the sample fluid is placing the insertable part to a mouth of a user. This way fluid to be used as the sample fluid can be collected from a user. The setup is beneficial as it eliminates for example spitting which is unreliable as it is difficult to aim to the elongated body of apparatus not to mention to for example a small input of lateral flow tests.

Optionally, the collecting further comprises:
contacting a pre-assembled insertable part with the sample fluid, wherein the pre-assembled insertable part is pre-assembled inside of the volume of the hollow elongate body; and
deforming the pre-assembled insertable part with the created pressure difference to deliver at least a part of the sample fluid to the at least one sensor.

Optionally, the method further comprises analysing the indicator value to determine a significance of the amount of the at least one molecule in the sample fluid.

### DETAILED DESCRIPTION OF DRAWINGS

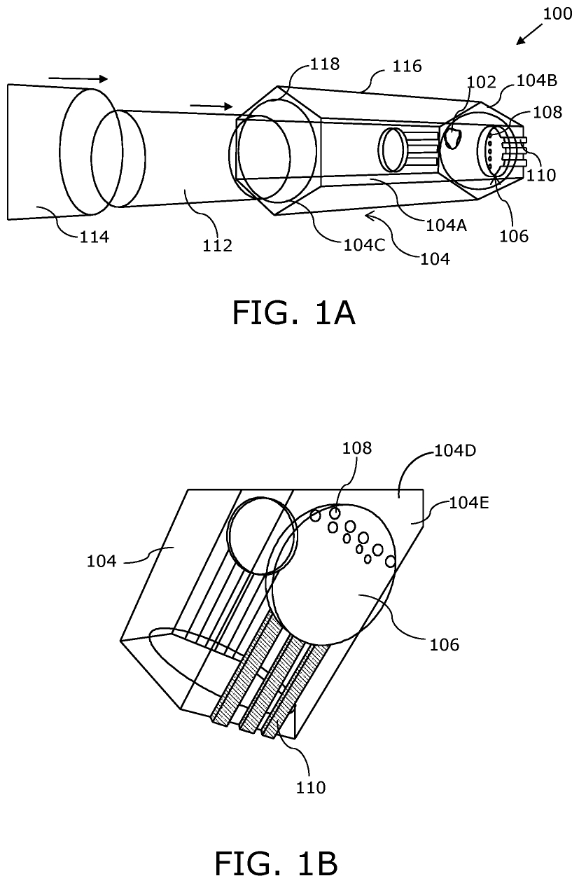

Referring to FIG. 1A, there is shown a perspective view of an apparatus 100 for collecting a sample fluid 102, in accordance with an embodiment of the present disclosure. The apparatus 100 comprises a hollow elongate body 104. Herein, at least one sensor 106 is coupled to the hollow elongate body 104 having a volume 104A, comprising a bottom 104B and an inlet 104C opposite to the bottom 104B of the volume 104A, wherein the volume 104A of the hollow elongate body 104 is configured to receive the sample fluid 102 via the inlet 104C. Moreover, the apparatus 100 comprises at least one sensor 106, coupled to the hollow elongate body 104, wherein the at least one sensor 106 has at least one opening 108 for receiving at least a part of the sample fluid 102 from the volume 104A. As shown, the at least one sensor 106 is positioned close to the bottom 104B of the hollow elongate body 104. Furthermore, the apparatus 100 comprises a plurality of electrical connectors 110, arranged with the hollow elongate body 104, connected to the at least one sensor 106. Optionally, the apparatus 100 may further comprise an inserting part 112 and a pressing part 114 respectively. The insertable part 112 is configured to be inserted at least partly into the volume 104A of the hollow elongate body 104, through the inlet 104C thereof. The pressing part 114 configured to drive the insertable part 112 into the volume 104A of the hollow elongate body 104. As shown, the hollow elongate body 104 may have a hexagonal shaped outer cross-section 116, with a circular shaped inner cross-section 118.

Referring to FIG. 1B, there is shown an enlarged view of at least one sensor 106 coupled with the hollow elongate body 104, in accordance with an embodiment of the present disclosure. Herein, the at least one sensor 106 is coupled to the hollow elongate body 104 on an inner surface 104D of a side 104E of the hollow elongate body 104. Moreover, the at least one sensor 106 comprises at least one opening 108. Furthermore, the at least one sensor 106 is connected to a plurality of electrical connectors 110.

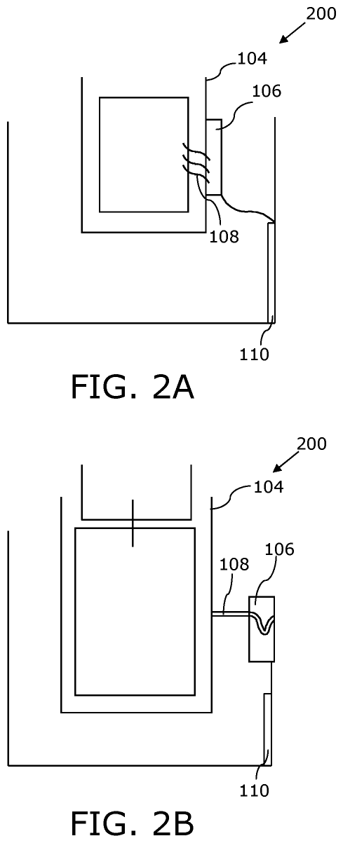

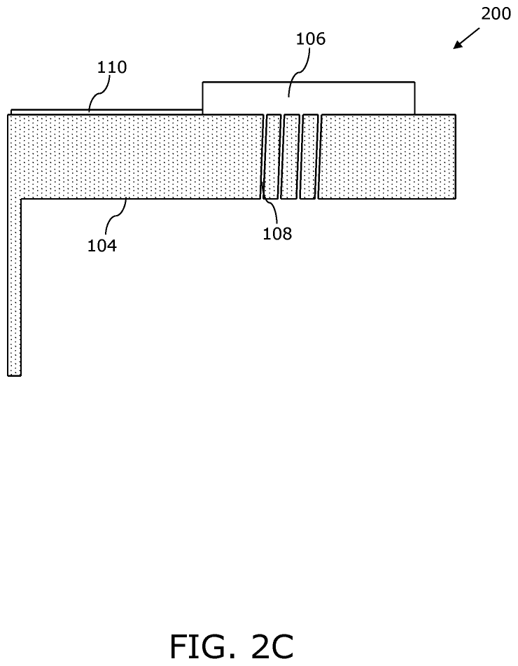

Referring to FIGS. 2A and 2B, there are shown schematic illustrations 200 of at least one opening 108 of at least one sensor 106 coupled with the hollow elongate body 104, in accordance with different embodiments of the present disclosure. As shown in FIG. 2A, at least one opening 108 of the at least one sensor 106 is implemented as a porous membrane. As shown in FIG. 2B, at least one opening 108 of the at least one sensor 106 is implemented as microfluidic channels.

Referring to FIG. 2C, there is shown a cross-sectional view of FIG. 2B, at least one sensor 106 is coupled to a hollow elongate body 104, in accordance with an embodiment of the present disclosure. As shown, the at least one sensor 106 is connected to a plurality of electrical connectors 110 arranged with the hollow elongate body 104. Moreover, the at least one sensor 106 has at least one opening 108 implemented as microfluidic channels.

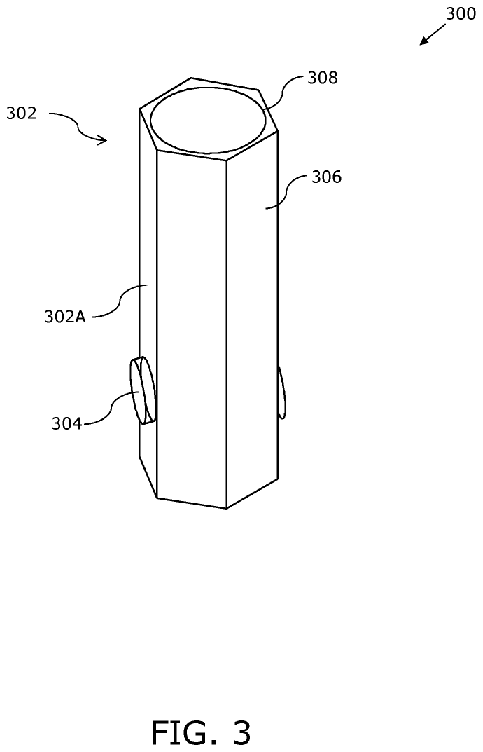

Referring to FIG. 3, there is shown a perspective view of an arrangement of at least one sensor coupled with the hollow elongate body 302, in accordance with another embodiment of the present disclosure. Herein, the at least one sensor 304 is coupled to the hollow elongate body 302 on an outer surface 302A of a side 302B of the hollow elongate body 302. As shown, the hollow elongate body 302 may have a hexagon shaped outer cross-section 306, with a circular shaped inner cross-section 308.

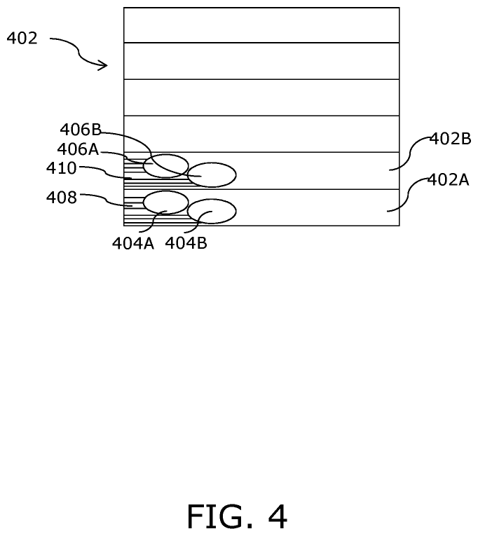

Referring to FIG. 4, there is shown a sheet 402 of fabrication material which can be wrapped to form a hollow elongate body, in accordance with an embodiment of the present disclosure. As shown, the sheet 402 divided into sections that form sides, such as the sides 402A and 402B.

Each of the sides 402A and 402B comprise 2 sensors 404A and 404B and 406A and 406B connected to a plurality of electrical connectors 408 and 410, respectively. The sheet 402 are printed, and are configured to be wrapped up to provide a hollow elongate body having a hexagonal shaped outer cross-section, with a hexagonal shaped inner cross-section. This is beneficial as manufacturing of the hollow elongated body with one or more sensors can be done in easy manner. The sheet 402 of fabrication material can be part of longer sheet from which sheet 402 are cut off. The sheet 402 can be then rolled as 3d object by welding (gluing or otherwise attaching the upper side (as in the figure) with the lower side. Indeed the outer surface of the apparatus is n-gonal prism. n-gonal prism refers to a prism which is polyhedron comprising an n-sided polygon base. Number of sides is 3 or higher.

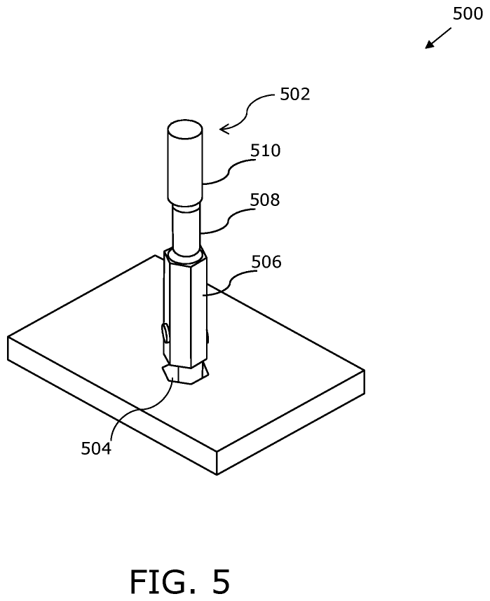

Referring to FIG. 5, there is shown a perspective view of a device 500 for collecting a sample fluid, in accordance with an embodiment of the present disclosure. As shown, the device 500 comprises the apparatus 502 and a reader part 504. Moreover, a cross-section of a hollow elongate body 506 and the reader part 504 is hexagonal in shape and a cross-section of an insertable part 508 and a pressing part 510 are cylindrical in shape.

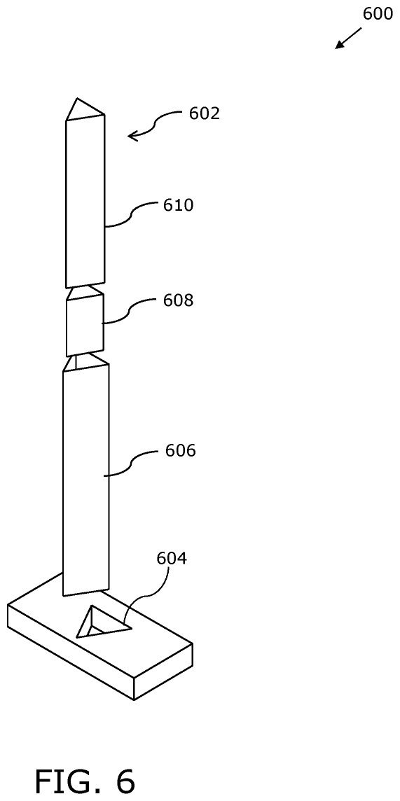

Referring to FIG. 6, there is shown a perspective view of a device 600 for collecting a sample fluid, in accordance with another embodiment of the present disclosure. As shown, the device 600 comprises the apparatus 602 and a reader part 604. Moreover, a cross-section of the reader part 604, a hollow elongate body 606, the insertable part 608 and the pressing part 610 is of triangular pyramid shape.

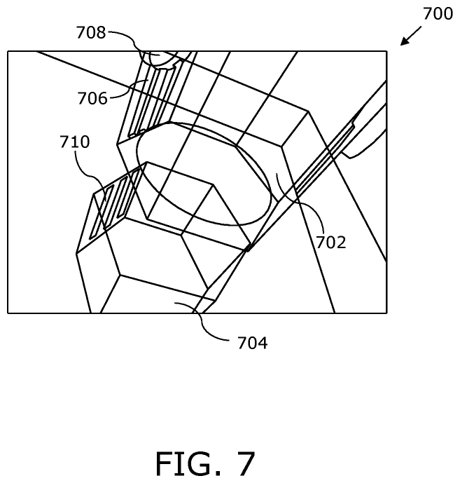

Referring to FIG. 7, there is shown a perspective view depicting the engagement of a hollow elongate body 702 of an apparatus with a reader part 704, in accordance with an embodiment of the present disclosure. Herein, the hollow elongate body 702 engages with the reader part 704. Furthermore, the plurality of electrical connectors 706 of the at least one sensor 708 connects with a plurality of base connectors 710 of the reader part 704.

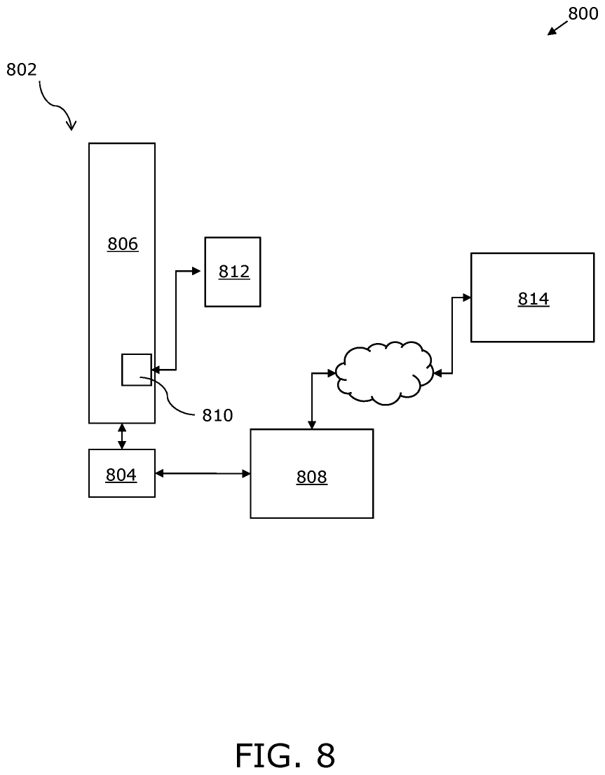

Referring to FIG. 8, there is shown an implementation environment 800 of a device 802 for collecting a sample fluid, in accordance with an embodiment of the present disclosure. As shown, the device 802 comprising a reader part 804 is configured to connect with an apparatus 806 and a controller 808. The controller 808 is configured to provide an electrical signal to at least one sensor 810 (associated with the apparatus 806) and read a related electrical signal from the at least one sensor 810, and use the provided and related electrical signal to form an indicator value of an amount of at least one molecule of interest in the sample fluid.

Moreover, the device 802 further comprises a measurement arrangement 812 configured to measure an amount of sample fluid supplied to the at least one sensor 810 and use the measured amount of the sample fluid to provide an instruction to a user of the device 802.

Furthermore, the device 802 comprises a communication interface (not shown) to provide the indicator value to a computing unit 814, wherein the computing unit 814 is configured to analyse the indicator value to determine a significance of the amount of the at least one molecule of interest in the sample fluid. Indeed the analysis can provide information of for example how many mmols/liter of molecule of interest was in the collected sample fluid.

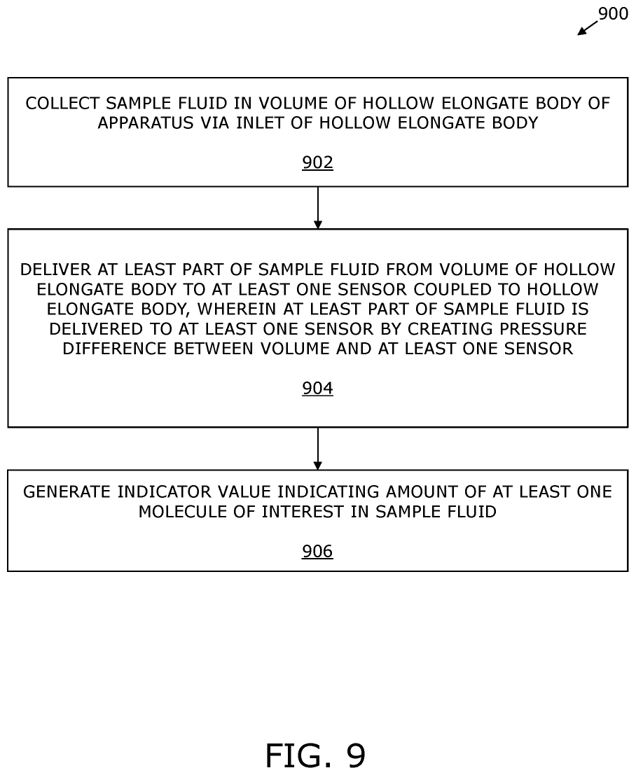

Referring to FIG. 9, there is shown a flowchart 900 depicting steps of a method for collecting a sample fluid, in accordance with an embodiment of the present disclosure. At step 902, the sample fluid is collected in a volume of a hollow elongate body of an apparatus, via an inlet of the hollow elongate body. At step 904, at least a part of the sample fluid is delivered from the volume of the hollow elongate body to the at least one sensor coupled to the hollow elongate body, wherein at least a part of the sample fluid is delivered to the at least one sensor by creating a pressure difference between the volume and the at least one sensor. At step 906, an indicator value indicating an amount of at least one molecule of interest in the sample fluid is generated.

The steps 902, 904 and 906 are only illustrative and other alternatives can also be provided where one or more steps are added, one or more steps are removed, or one or more steps are provided in a different sequence without departing from the scope of the claims herein.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Expressions such as “may” and “can” are used to indicate optional features, unless indicated otherwise in the foregoing. Reference to the singular is also to be construed to relate to the plural.

1. An apparatus for collecting a sample fluid, the apparatus comprising:
a hollow elongate body having a volume, comprising a bottom and an inlet opposite to the bottom of the volume, wherein the volume of the hollow elongate body is configured to receive the sample fluid via the inlet;
at least one sensor, coupled to the hollow elongate body, wherein the at least one sensor has at least one opening for receiving at least a part of the sample fluid from the volume; and
a plurality of electrical connectors, arranged with the hollow elongate body, connected to the at least one sensor.

2. The apparatus according to claim 1, wherein the at least one sensor is coupled on an inner surface of at least one side of the hollow elongate body.

3. The apparatus according to claim 1, wherein the at least one sensor is coupled on an outer surface of at least one side of the hollow elongate body.

4. The apparatus according to claim 1, wherein the at least one opening of the at least one sensor is implemented as holes, microfluidic channels, or a porous membrane.

5. The apparatus according to claim 1 further comprising an insertable part configured to be inserted at least partly into the volume of the hollow elongate body, through the inlet thereof, to deliver the sample fluid to the at least one sensor.

6. The apparatus according to claim 5, wherein the insertable part comprises a porous tissue for collecting the sample fluid.

7. The apparatus according to claim 1 further comprising a pressing part configured to drive the insertable part into the volume of the hollow elongate body, and to change pressure inside of the volume to deform the insertable part to deliver the sample fluid to the at least one sensor.

8. The apparatus according to claim 7, wherein the pressing part is attached to the insertable part.

9. The apparatus according to claim 1, wherein the at least one sensor is an electrochemical sensor adapted to change its electrical characters as a function of an amount of a first molecule of interest in the sample fluid.

10. The apparatus according to claim 9, wherein number of electrochemical sensors is at least two, of which a first one is adapted to change its electrical characters as the function of the amount of the first molecule of interest in the sample fluid and a second one is adapted to change its electrical characters as a function of an amount of a second molecule of interest in the sample fluid, and wherein the first molecule is different from the second molecule.

11. The apparatus according to claim 1 wherein the outer surface of the apparatus is n-gonal prism.

12. A device for collecting a sample fluid, the device comprising:
an apparatus for collecting the sample fluid wherein the apparatus comprises:
a hollow elongate body having a volume comprising a bottom and an inlet opposite to the bottom of the volume, wherein the volume of the hollow elongate body is configured to receive the sample fluid via the inlet;
at least one sensor coupled to the hollow elongate body, wherein the at least one sensor has at least one opening for receiving at least a part of the sample fluid from the volume; and
a plurality of electrical connectors arranged with the hollow elongate body, connected to the at least one sensor; and
a reader part having a plurality of base connectors to connect with a plurality of electrical connectors of at least one sensor of the apparatus, and a controller configured to
provide an electrical signal to the at least one sensor and read a related electrical signal from the at least one sensor via the plurality of base connectors, and
use the provided and related electrical signal to form an indicator value of an amount of at least one molecule of interest in the sample fluid.

13. The device according to claim 12 further comprising a measurement arrangement configured to measure an amount of sample fluid supplied to the at least one sensor and use the measured amount of the sample fluid to provide an instruction to a user of the device.

14. The device according to claim 12 or 13, wherein the device comprises a communication interface to provide the indicator value to a computing unit, wherein the computing unit is configured to analyse the indicator value to determine a significance of the amount of the at least one molecule of interest in the sample fluid.

15. A method for collecting a sample fluid, the method comprising:
collecting the sample fluid in a volume of a hollow elongate body of an apparatus via an inlet of the hollow elongate body;
delivering at least a part of the sample fluid from the volume of the hollow elongate body to the at least one sensor coupled to the hollow elongate body, wherein at least a part of the sample fluid is delivered to the at least one sensor by creating a pressure difference between the volume and the at least one sensor; and
generating an indicator value indicating an amount of at least one molecule of interest in the sample fluid.

16. The method according to claim 15, wherein the collecting further comprises:
contacting an insertable part with the sample fluid;
inserting the insertable part into the volume of the hollow elongate body via an inlet of the hollow elongate body to create a pressure difference between the volume and the at least one sensor; and
deforming the insertable part with the created pressure difference to deliver at least a part of the sample fluid to the at least one sensor.

17. The method according to claim 15, wherein the collecting further comprises:
contacting a pre-assembled insertable part with the sample fluid, wherein the pre-assembled insertable part is pre-assembled inside of the volume of the hollow elongate body; and
deforming the pre-assembled insertable part with the created pressure difference to deliver at least a part of the sample fluid to the at least one sensor.

18. The method according to claim 15, further comprising analysing the indicator value to determine a significance of the amount of the at least one molecule in the sample fluid.
